(19) United States
(12) Patent Application Publication
(10) Pub. No.: US 2026/0273269 A1
(43) Pub. Date: Sep. 17, 2026

(54) A SYSTEM COMPRISING A CONTROLLER AND AN ELECTRICAL STIMULATION SYSTEM

(71) Applicant: Matrix Muscle Support AB, Solna (SE)

(72) Inventors: Paul Wilhelm Ackermann, Enebyberg (SE); Robin Assar Juthberg, Solna (SE)

(21) Appl. No.: 19/166,785

(22) PCT Filed: Mar. 19, 2024
(86) PCT No.: PCT/SE2024/050249

§ 371 Date: Sep. 18, 2025

(30) Foreign Application Priority Data
Mar. 20, 2023 (GB) 2304024.9

(51) Int. Cl. A61N 1/04; A61N 1/36

(52) U.S. Cl. CPC A61N 1/0476; A61N 1/36031; A61N 1/36034

(57) ABSTRACT

The present disclosure provides for a controller and method for providing signalling to an ES system to operate an electrode array in a detection mode, to cause the ES system to provide a plurality of electrical parameter detection bursts, wherein each electrical parameter detection burst is configured to not provide for muscular stimulation; and detect one or more detection parameters indicative of the degree of movement of the muscle associated with each electrical parameter detection burst, wherein each electrical parameter detection burst is provided by applying a potential difference between different pairs of the plurality of electrodes, wherein the controller is configured to calculate the electrical parameter between each pair of detection electrodes based on the one or more detection parameters. The disclosure also relates to an associated electrical stimulation system and a computer readable medium.

### FIELD

The present disclosure relates to a controller for an electrical stimulation(ES) system and a corresponding ES system.

### BACKGROUND

Inactivity is developing into the biggest health challenge of current times. This problem is especially severe for medical patients who are immobilised. Immobilisation and muscle inactivity lead to major medical disorders, such as muscle wasting, diabetes, pain, overweight, oedema, deep vein thrombosis and pulmonary embolism which may all lead to suffering and death. These medical disorders pose immense costs on healthcare but are potentially preventable.

Treatment of immobilisation requires mobilisation, i.e., physical activity, which may not be performed in the right amount and is not always possible for some people. In summary, low efficiency of interventions is caused by low compliance to treatment. One treatment to activate immobilised muscles is neuromuscular electrical stimulation (NMES), an example of an electrical stimulation(ES) technique, which is used by physiotherapists and, to a lesser extent, by end users to stimulate inactive skeletal muscles.

Effective application of NMES requires the correct positioning of electrodes on so-called motor points to achieve the most comfortable and least energy-consuming muscle stimulation. While motor points tend to be in similar locations across individuals, there is still a large variation in the exact position between individuals. Furthermore, even the individual motor points of a single user may vary in position from one time to another. A matrix of electrodes can be used to stimulate a nerve or muscle at a body location. Measuring such stimulation requires adding sensors, such as flex sensors or other sensors to detect movement at the body location. Balancing sensitivity with accuracy and flexibility in the application of stimulation can be challenging, as adding appropriate sensors can take up space that could otherwise be used for electrodes in the electrode matrix.

### SUMMARY

According to a first aspect of the present disclosure, there is provided a controller for providing signalling to, and receiving signalling from, at least one electrical stimulation, ES, system, the ES system comprising an electrode array comprising a plurality of electrodes for applying an electrical stimulation to a muscle of a user and wherein the electrodes are further configured to measure an electrical parameter between any one electrode of the electrode array and any other one electrode of the electrode array, wherein the controller is configured to: provide signalling to the ES system to operate the electrode array in a detection mode during a detection period, the signalling causing the ES system to provide a plurality of electrical parameter detection bursts, wherein each electrical parameter detection burst comprises one or more detection pulses and wherein each electrical parameter detection burst is configured to not provide for muscular stimulation; and detect one or more detection parameters indicative of a degree of movement of the muscle associated with each electrical parameter detection burst, wherein each electrical parameter detection burst is provided by applying a potential difference between different pairs of the plurality of electrodes, wherein a pair of electrodes refers to the combination of a first detection electrode and a second detection electrode selected from any electrode of the electrode array such that, when the controller provides a subsequent electrical parameter detection burst, the corresponding electrode pair for that subsequent electrical parameter detection burst comprises a pair of any two electrodes of the electrode array except for any electrode pair which would correspond to a previously provided electrical parameter detection burst of the detection period, wherein the controller is configured to calculate the electrical parameter between each pair of detection electrodes based on the one or more detection parameters.

It will be appreciated that not providing for muscular stimulation refers to applying an electrical parameter detection burst at an intensity sufficient to cause muscular activation. It will be appreciated that a degree of movement of the muscle is an amount of movement of the muscle and may include zero movement. That is, the detection parameters can indicate whether or not, or to what degree, the muscle has moved. In some embodiments, the degree of movement of the muscle or body is a response to muscular stimulation caused by a stimulation pulse.

In one or more embodiments, the controller may be configured to provide signalling to the ES system to operate the electrode array in a stimulation mode, the signalling causing the ES system to provide a plurality of stimulation pulses separated by rest periods wherein: each stimulation pulse comprises applying a stimulating potential difference between a first stimulation electrode and a second stimulation electrode of the electrode array such that the stimulating potential difference is configured to provide for stimulation of the muscle; and each rest period comprises a period of time which is absent of stimulation pulses.

In one or more embodiments, a detection period may occur during a rest period such that the one or more detection parameters are measured between the application of stimulation pulses.

In one or more embodiments, the controller may be configured to provide signalling to the ES system to cause the ES system to provide an electrical parameter detection burst between at least 50% of the pairs of detection electrodes that can be defined within the electrode array and correspondingly detect one or more detection parameters indicative of the degree of movement of the body associated with each electrical parameter detection burst.

In yet other alternative embodiments, the controller may be configured to provide signalling to the ES system to cause the ES system to provide an electrical parameter detection burst between at least 30%, at least 40%, at least 60%, at least 70%, at least 80%, at least 90% or at least 100% of the pairs of detection electrodes that can be defined within the electrode array and correspondingly detect one or more detection parameters indicative of the degree of movement of the body associated with each electrical parameter detection burst. By taking a large number of measurements between different pairs of electrodes that can be defined within the array, one effectively creates a corresponding number of sensors which can provide improved accuracy, reliability and location specificity compared to, instead, using a small handful of flex sensors, for example.

In one or more embodiments, the controller may be further configured to determine, based on the electrical parameters calculated between each pair of detection electrodes, whether to provide signalling to the ES system to operate the electrode array in the detection mode or in an alternate mode.

In one or more embodiments, a detection parameter may be the current between a pair of electrodes during the respective electrical parameter detection burst.

In one or more embodiments, a calculated electrical parameter may be at least one of: an electrical resistivity; an electrical resistance; an electrical conductivity; an electrical conductance; an electrical impedance; and an electrical admittance.

In one or more embodiments, the controller may be further configured to generate an electrical parameter map representative of a spatial distribution of the electrodes of the electrode array, wherein the electrical parameter map comprises a plurality of elements arranged in a grid and wherein each element comprises an element value based on one or more electrical parameters calculated by the controller.

In one or more embodiments, each element of the electrical parameter map may correspond to an electrode of the electrode array, and the element value of an element of the electrical parameter map is based on a sum of each calculated electrical parameter that used the corresponding electrode.

In one or more embodiments, each element of the electrical parameter map may correspond to an electrode of the electrode array, and the element value of an element of the electrical parameter map is based on an average of each calculated electrical parameter that used the corresponding electrode.

In one or more embodiments, the controller may be configured to obtain an electrical parameter map for electrodes of the electrode array obtained during a current detection period and wherein the element values of the electrical parameter map are based on a difference between element values calculated during the current detection period and one of: an element value calculated during a preceding detection period taken at a time earlier than the current detection period; and predefined element values.

In one or more embodiments, each element of the electrical parameter map may correspond to a pair of electrodes of the electrode array, and the element value of an element of the electrical parameter is based on the difference between an electrical parameter calculated between the pair during the electrical parameter detection burst, and an electrical parameter calculated between the pair during a previous electrical parameter detection burst.

In one or more embodiments, the controller may be further configured to determine, based on the electrical parameter map, whether to provide signalling to the ES system to operate the electrode array in the detection mode or in an alternate mode.

In one or more embodiments, the electrical parameter detection burst may comprise a plurality of detection pulses each with a pulse frequency of between 10 Hz and 100 MHz.

In one or more embodiments, the current supplied between each pair of detection electrodes for detection of a detection parameter may be no more than 0.1 mA.

In one or more embodiments, the controller may be configured to, during the stimulation period, cause the ES system to apply a plurality of skin resistance reduction pulses, wherein the skin resistance reduction pulses comprise a higher frequency and a lower intensity compared to the plurality of stimulation pulses.

In one or more embodiments, a plurality of the skin resistance reduction pulses may be superimposed over a plurality of the stimulation pulses.

In one or more embodiments, the skin resistance reduction pulses may: carry a current of no more than 0.1 mA per pulse; and have a frequency between 1 and 100 000 Hz.

In one or more embodiments, the controller may be configured to, while operating in the stimulation mode, cause the ES system to apply a plurality of sensory blocking pulses, wherein the sensory blocking pulses have a lower intensity than the intensity of the stimulation pulses.

In one or more embodiments, the sensory blocking pulses may have a frequency between 0.1 and 150 Hz.

According to a second aspect of the present disclosure, there is provided an electrical stimulation, ES, system for receiving signalling from and providing signalling to a controller of the first aspect.

In one or more embodiments, the ES system of the second aspect may further comprise a garment wherein the garment provides a support for the relative arrangement of the electrodes in the electrode array and is configured to distribute at least the electrodes over at least a body part of the user.

According to a third aspect of the present disclosure, there is provided a computer readable medium comprising computer program code configured to cause a controller to operate according to the first aspect or the second aspect.

According to a fourth aspect of the present disclosure, there is provided a method for providing electrical stimulation, ES, to a muscle of a user using at least one ES system, the ES system comprising a controller and an electrode array comprising a plurality of electrodes for applying the electrical stimulation to the muscle and wherein the electrodes are further configured to measure an electrical parameter between any one electrodes of the electrode array and any other one electrode of the electrode array, the method comprising the steps of: the controller providing signalling to the ES system to operate the electrode array in a detection mode during a detection period, the signalling causing the ES system to provide a plurality of electrical parameter detection bursts during at least one detection period wherein each electrical parameter detection burst comprises one or more detection pulses and wherein each electrical parameter detection burst is configured to not provide for muscular stimulation; and the controller detecting one or more detection parameters indicative of a degree of movement of the muscle associated with each electrical parameter detection burst, wherein each electrical parameter detection burst is provided by applying a potential difference between different pairs of the plurality of electrodes, wherein a pair of electrodes refers to the combination of a first detection electrode and a second detection electrode selected from any electrode of the electrode array such that, when the controller provides a subsequent electrical parameter detection burst, the corresponding electrode pair for that subsequent electrical parameter detection burst comprises a pair of any two electrodes of the electrode array except for any electrode pair which would correspond to a previously provided electrical parameter detection burst of the detection period, and the controller calculating the electrical parameter between each pair of detection electrodes based on the one or more detection parameters.

In one or more embodiments, the method of the fourth aspect may further comprising the step of: the controller providing signalling to the ES system to operate the electrode array in a stimulation mode, the signalling causing the ES system to provide a plurality of stimulation pulses separated by rest periods wherein: each stimulation pulse comprises applying a stimulating potential difference between a first stimulation electrode and a second stimulation electrode of the electrode array such that the stimulating potential difference is configured to provide for stimulation of the muscle; and each rest period comprises a period of time which is absent of stimulation pulses.

According to a fifth aspect of the present disclosure, there is provided a controller for providing signalling to, and receiving signalling from, at least one system, the system comprising an electrode array comprising a plurality of electrodes, and wherein the electrodes are further configured to measure an electrical parameter between any one electrode of the electrode array and any other one electrode of the electrode array, wherein the controller is configured to: provide signalling to the system to operate the electrode array in a detection mode during a detection period, the signalling causing the system to provide a plurality of electrical parameter detection bursts, wherein each electrical parameter detection burst comprises one or more detection pulses and wherein each electrical parameter detection burst is configured to not provide for muscular stimulation; and detect one or more detection parameters indicative of a degree of movement of the muscle associated with each electrical parameter detection burst, wherein each electrical parameter detection burst is provided by applying a potential difference between different pairs of the plurality of electrodes, wherein a pair of electrodes refers to the combination of a first detection electrode and a second detection electrode selected from any electrode of the electrode array such that, when the controller provides a subsequent electrical parameter detection burst, the corresponding electrode pair for that subsequent electrical parameter detection burst comprises a pair of any two electrodes of the electrode array except for any electrode pair which would correspond to a previously provided electrical parameter detection burst of the detection period, wherein the controller is configured to calculate the electrical parameter between each pair of detection electrodes based on the one or more detection parameters. In one or more examples, a controller of the fifth aspect may be a controller for a sensing system where the sensing system is configured to sense movement of a muscle (i.e., stimulation of the muscle) whether movement of the muscle was induced by purposeful user movement, involuntary user movement or whether that movement is instigated by an external stimulation device. The following description provides the disclosure in the context of a stimulation system, however, it will be appreciated that any parts of the disclosure which are not inherently related to causing stimulation may be equally implemented in a sensing system, such as a sensing system of the fifth aspect, for sensing muscle movement.

Although the present disclosure can be described in in relation to NMES treatment, as discussed in the background, other applications are also envisioned. There is a need for accurate, low-cost motion detection in many industries.

One example is the gaming and simulation industry, in particular virtual reality (VR) and assisted reality (AR) gaming. Embodiments of the present disclosure can be used to capture the motion of one or more areas of a user's body. The motion capture data is then processed and used as an input into the control feedback loop of a gaming interface. This provides the user with a more immersive gaming experience. Embodiments of the present disclosure may be integrated into a garment or other wearable device, which the user wears while playing the game. Some embodiments of the present disclosure may be configured to provide mild electrical stimulation to a user as part of the control feedback loop of the gaming interface. For example, if an area of the user's avatar in the game is injured, mild electrical stimulation may be applied to the corresponding area of the user's body to alert the user to the injury.

In another example, embodiments of the present disclosure may be used in conjunction with motion augmentation systems such as powered exoskeletons. Exoskeletons are being increasingly used in military, industrial and medical applications to provide a user's body with structural support. They can enhance a user's strength and endurance, or assist the movements of an injured or disabled user. Embodiments of the present disclosure can be used to capture the motion of one or more areas of a user's body. The motion capture data is then processed and used as an input into the control feedback loop of a powered exoskeleton, allowing the user to control the exoskeleton using their body. In some embodiments, the system is configured to provide mild electrical stimulation to the user's body, providing feedback from the powered exoskeleton. For example, a user may be alerted to a contact force applied to the exterior of the exoskeleton by applying mild electrical stimulation to the corresponding area of the user's body. The electrode array can be integrated into flexible materials and garments, as used by the latest generations of “soft” exoskeletons (also known as powered clothing).

### BRIEF DESCRIPTION OF THE DRAWINGS

One or more embodiments will now be described by way of example only with reference to the accompanying drawings in which:

FIG. 1 shows an example embodiment of a system of the present disclosure comprising a controller and an ES system;

FIG. 2 shows an example representation of motor points on the calf of a user;

FIG. 3 shows an example embodiment of an electrode array on the calf of a user;

FIG. 4 shows an example embodiment of an electrode array, with individual electrical parameter detection bursts illustrated figuratively as dotted lines;

FIG. 5 is a voltage against time trace plot showing an exemplary plurality of a stimulation pulses and plurality of detection bursts between a pair of electrodes;

FIG. 6 shows an example embodiment of an electrode array including group electrodes;

FIGS. 7(a), (b) and (c) are voltage against time trace plots depicting a skin resistance reduction pulse between a pair of electrodes, independently and in conjunction with a stimulation pulse;

FIG. 8 shows example voltage against time trace plots depicting a sensory blocking pulse in conjunction with a stimulation pulse;

FIG. 9 shows an example method for providing electrical stimulation to a muscle of a user using at least one electrical stimulation(ES) system according to the present disclosure;

FIG. 10 is a flow chart illustrating an exemplary method for performing detection; and

FIG. 11 shows an example embodiment of a computer-readable storage medium.

### DETAILED DESCRIPTION

As shown in FIG. 1, in the present disclosure there is described a system 100 comprising a controller 101 and an electrical stimulation, ES, system 102. The ES system 102 comprises an electrode array 104 and one or more sensors 105. In one or more embodiments, the ES system 102 may further comprise a garment 103. In one or more embodiments, the ES system 102 may comprise a plurality of garments 103, wherein each garment comprises a corresponding electrode array 104 and one or more sensors 105 or a single garment may comprise a plurality of electrode arrays 104 and associated one or more sensors 105. Within a single garment, each electrode array 104 may form an independent ES system 102 with its own controller.

The controller 101 may be any appropriate electronic controller 101 that is configured to provide signalling to and receive signalling from the ES system 102 or any component of the ES system 102. The controller 101 may comprise at least one processor and at least one memory including computer program code. The at least one memory and the computer program code may be configured to, with the at least one processor, cause the controller 101 to provide for signalling to the ES system 102. The controller 101 may also be configured to receive signalling from the ES system 102 and process the received signalling such that the controller 101 can act on the information comprised within the signalling. The actions that the controller 101 can take based on the received signalling will be described in greater detail below. It will be appreciated that any suitable means may be used by the controller 101 to provide for provision and receipt of signalling. For example, the controller 101 may be electrically coupled to the ES system 102 such that signals are transmitted directly via physical conducting means such as wires, conductive tracks, conductive fibres or conductive fabrics. Alternatively, the controller 101 may provide for signalling to cause a wireless transceiver to provide for the transmission and receipt of signalling to the ES system 102 and the ES system 102 may comprise a corresponding transceiver configured to both transmit and receive signalling. In such examples, the ES system 102 may comprise an ES controller (not shown) configured to interpret the signalling received from the controller 101.

In some examples, the controller 101 may be incorporated into one of the one or more garments 103 or may be a remote device such as a hip-mounted control box or a computing device such as a mobile phone, tablet computer, laptop computer or personal computer (PC) that may provide for communication with the one or more garments 103 of the ES system 102.

In one or more embodiments, the controller 101 may comprise a coordination controller and one or more ES system controllers. In such embodiments, each ES system controller sends and receives signals to control a single ES system 102, and sends and receives signals up the coordination controller. The coordination controller sends high-level control signals to the ES system controllers, which then generate low-level control signals for the ES system 102. For example, the coordination controller may instruct all of the ES system controllers to switch into a detection mode, causing the ES system controllers to begin generating signals to measure detection parameters between pairs of electrodes in their electrode arrays 104.

In one example, a user wears a garment covering their legs, which includes a plurality of discrete ES systems each including an ES system controller. One ES system includes an electrode array 104 positioned over the right leg, and another ES system includes an electrode array 104 positioned over the left leg. The coordination controller may be a mobile phone, tablet or any other mobile device, which communicates wirelessly with each of the ES system controllers. The coordination controller performs calculations and determines the operating mode of the ES systems 102.

Electrical stimulation(ES) covers a range of techniques which involve providing stimulation to one or more muscles or nerves at a target body location by the application of one or more electrical signals or pulses. One such example of ES is neuromuscular electrical stimulation (NMES), however, it will be appreciated that any suitable ES technique may be used. Other examples of suitable electrical stimulation techniques may include, but are not limited to, electrical muscle stimulation (EMS), Russian electrical stimulation, functional electrical stimulation (FES) and transcutaneous electrical nerve stimulation (TENS).

In one or more examples, the plurality of electrodes of the electrode array 104 may be configured to be connected directly to the user by any suitable means. The electrode array may comprise both stimulation electrodes configured to provide for stimulation of one or both of muscles and nerves and detection electrodes configured to detect the electrical parameters indicative of stimulation of muscles or nerves, For example, a plurality of individual electrode pads may be attached to the user and those electrodes together define the array of electrodes 104. In such embodiments, the controller 101 may be connected to the electrodes of the electrode array 104 in any suitable manner. In yet other examples, one or more of the electrodes of the electrode array 104 may provide the functionality of both stimulation electrodes and detection electrodes. In other examples, a garment 103 may provide support for the relative arrangement of electrodes of the electrode array 104. The garment 103 may, for example, be an article of clothing, a patch, a wrapping or an adhesively attached pad configured to be worn by the user.

It will be appreciated that, herein, the user is defined as the person or animal to which electrical stimulation is to be applied. This does not exclude one or more other people assisting the user, such as medical practitioners. It may even be that the user is not conscious and not operating the system 100 at all but that others, such as medical practitioners, are providing any necessary input to the system.

In examples where the ES system 102 comprises a single garment 103, the system 100 as a whole may be entirely incorporated into a garment 103 itself such that the controller 101 comprises a microprocessor embedded into garment 103 along with the electrode array 104 and the one or more sensors 105 of the ES system 102. In other examples, the overall system 100 may comprise a plurality of garments 103 wherein each of the electrode array 104 and the one or more sensors 105 of each garment is in signalling-communication with the controller 101. In embodiments where the ES system 102 comprises a plurality of garments 103, the controller 101 may be incorporated into one of the garments 103 or may be incorporated into a device remote from any of the garments 103 of the ES system 102.

The technique of ES can be used to prevent several undesirable medical afflictions, as discussed already, or to improve bodily functions, Correctly applied ES helps to reduce the impact and likelihood of these afflictions taking effect by several mechanisms including: stimulating the muscles and thereby causing them to work and either get stronger or maintain their current strength; by forcing the movement of blood through the veins which also improves arterial circulation; activating the nervous system; releasing growth factors; and releasing anti-blood clotting factors. The additional assistance of moving the blood may be particularly beneficial and helpful for some users for whom blood circulation is below desirable levels.

A garment 103 herein refers to an article which can be worn on a human or animal body where the human or animal wearing the garment 103 is defined as the user of the ES system 102. It will be appreciated that garments 103 may include articles of clothing such as socks, gloves, tights, boxer shorts, long johns, vests or other articles of clothing. Alternatively, the garment 103 may be a bandage, a patch, a cast or another medical wrapping or support garment which may be applied to a human or animal body for prolonged periods and during normal activities of the user. The garments 103 referred to herein are configured to provide for support for the relative arrangement of electrodes of an electrode array 104 and one or more sensors 105 in order to distribute at least the electrodes over at least part of a body of a user. The garment 103 may be configured to distribute the electrodes of the electrode array 104 such that each electrode is in contact with the skin of a user such that the application of two different voltages at two different electrodes of the electrode array 104 causes stimulation of a muscle or its innervation below the skin, if present. In the case where a muscle or its innervation is located beneath the skin with which the electrodes are in electrical communication, the muscle may be caused to contract in response to the electrical stimulation. The stimulation of a muscle can be performed by its innervation, which refers to the electrical stimulation of an adjacent or distant nerve connected to the muscle. Muscular stimulation via nerve innervation may alternatively be referred to as indirect muscular stimulation while direct stimulation of the muscle may be referred to as direct muscular stimulation.

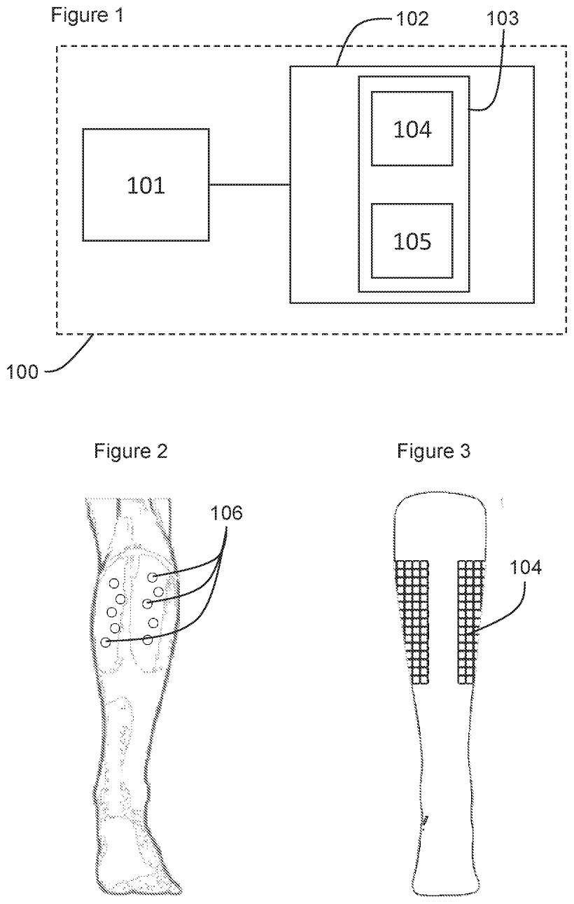

FIG. 2 shows an example of the leg of a human user. Each muscle in the body comprises one or more motor points 106. The motor points 106 are defined as the areas of the skin above the muscle or its innervation which require the lowest electrical stimulation to cause a muscle twitch or contraction. Herein a motor point 106 of a muscle may refer to a point that connects to a nerve that, when electrical stimulation is applied thereto, provides for contraction of the muscle by innervation. For example, the calf muscle may comprise a plurality of different motor points 106. Power can be saved in an ES device by accurately targeting motor points 106 of a user for electrical stimulation, thereby increasing the lifetime of a system 100 of the present disclosure before recharging is required. Further, it has been found that discomfort of a user is reduced when the motor points 106 are electrically stimulated as compared to stimulating non-motor points. Electrical stimulation at a pair of motor points, as defined herein, may also provide for increased blood flow in a user by providing for stimulation of a muscle. In some examples, the optimum points for stimulation may not be optimum points for achieving the greatest possible muscle contraction or for the lowest possible current requirement for stimulation. Nevertheless, since such selected points provide for an optimum desired result, such points will also be referred to herein as motor points. It may be particularly advantageous to provide ES stimulation via those electrodes in closest proximity to the motor points 106, as opposed to only placing a single electrode at a motor point 106 while a second electrode is placed at a non-motor point position. It is possible to provide for the identification of the optimal pairs of points for the provision of electrical stimulation to cause the muscle to contract. This may provide for the points on the body which are most comfortable for the user to have stimulated and which use the least energy to cause a desirable degree of contraction of the muscle. In some cases, one or both of these points may not be identified as a motor point 106 using traditional methods because traditional methods operate by manually searching for a single point relative to a fixed reference point such as by using motor point pens. In contrast, the system 100 may define motor points by using a motor point scan mode to search for one or more optimum pairs of points. That is, the motor points as defined herein will be those points which provide for an advantageous result in terms of one or more desired stimulation parameters. The one or more desired stimulation parameters may include, but are not limited to: maximum muscle contraction; lowest current required for a desired level of contraction; and maximum induced blood flow. As such, one may consider the motor points herein as optimum stimulation points for achieving a desired effect.

In one or more examples, it may be the case that a location over a muscle that is identified as a motor point using a traditional motor point identification technique, such as by using a motor point pen, may not be identified as a motor point using the approach disclosed herein. Traditional approaches that, in some cases, reuse a same standard reference electrode for each measurement in order to find a single motor point may not provide the same flexibility in identifying optimal pairs of locations. In fact, in some examples, motor points identified by a motor point scan mode may select two entirely different locations for motor points compared to traditional approaches.

FIG. 3 shows an example leg of a user and the arrangement of an electrode array 104 thereon. The electrode array 104 comprises a plurality of electrodes which are configured to be, when in use, in electrical contact with the skin of a user. It will be appreciated that this may generally mean that the electrodes are in direct physical contact with the skin of the user but that in other embodiments, one or more conductive materials may be disposed between each electrode and the skin in use.

The electrode array 104 may be arranged in any manner which is suitable for providing stimulation to a plurality of different points on the muscle. In one or more embodiments, the electrodes may be arranged in a matrix arrangement, as shown in FIG. 3. In the matrix arrangement, the electrodes may be arranged in a regular grid pattern in order to provide for comprehensive coverage of the muscle being targeted. Each electrode in the array of electrodes 104 may be individually addressable so that a voltage can be applied at each electrode in isolation from each other electrode. Each electrode in the electrode array 104 may be electrically isolated from each of the other electrodes when the electrodes are not placed in electrical contact with a conductive medium such as skin and, when in contact via the skin, a high resistivity contact is made therebetween. When an electrode does not have a voltage applied thereto as a result of signalling from the controller 101, the electrode may be uncoupled from any voltage source or ground of the ES system 102 such as by the opening of a switch. This may remove any chance that a current would flow between the wrong electrodes. The isolation of each of the unconnected electrodes may be controlled by the controller 101. The array of electrodes 104 may comprise, for example, more than 10, 20, 50, 100, 225 or any other number of electrodes.

The controller 101 may provide signalling to the ES system 102 to selectively operate the electrode array 104 in one of a plurality of modes, such as in a detection mode, a stimulation mode, a motor point scan mode or a calibration mode. In any of these three modes, a potential difference is applied between at least two different electrodes in the electrode array 104. The application of an electrical potential difference between these two electrodes can be configured to cause stimulation of one a muscle, either directly or indirectly, at a target body location wherein the target body location is a location at or between the two electrodes. If alternative parameters are selected, the application of the electrical potential difference between these two electrodes can be configured to avoid causing stimulation of a muscle at the target body location. For example, when operating in the detection mode, the controller 101 may be configured to cause the electrical stimulation system 102 to avoid stimulating a muscle at the target body location by selecting appropriate parameters. When operating in the motor point scan mode, the controller 101 may be configured to cause the electrical stimulation system 102 to stimulate a muscle to a lesser degree than in the stimulation mode. When operating in the stimulation mode, the controller 101 may be configured to cause the electrical stimulation system 102 to stimulate a muscle at the target body location to a greater degree than in the motor point scan mode by applying a plurality of stimulation pulses.

The voltages may be applied at opposing polarities which is to say that one voltage is a positive voltage while the second is a negative voltage relative to a reference voltage such as a ground voltage. The reference voltage may be considered to be a voltage part way between the two different voltages applied to the first and second electrodes and, as such, the voltages may be at opposing polarities. It will be appreciated that, relative to another reference voltage, the voltages may be measured as having other values, such as 0 V and +5 V. It will be appreciated that, no matter the reference voltage selected, it is the presence of an electrical potential difference between the electrodes which provides for the electrical stimulation. It may be considered that electrodes that do not have a voltage applied to them as a result of signalling provided by the controller 101 are substantially at a reference voltage such as the ground voltage, or those electrodes may be disconnected from the circuit entirely. In some examples, in order to create the electrical potential difference between the selected electrodes, the voltages applied to the electrodes may be applied contemporaneously.

The controller 101 can provide signalling to the ES system 102 to selectively operate the electrode array 104 in a detection mode, wherein the application of an electrical potential difference between pairs of electrodes can be used to detect body movement. This is done by sending low-intensity electrical pulses between two electrodes in the electrode array 104 through or across the user's body. The pathways taken by the pulses can be characterised by detection parameters. As the body moves, the relative position of the electrodes may change relative to each other, and the electrodes may also move relative to the body itself (for example by sliding over the skin). This changes the pathways taken by detection pulses between those electrodes, which in turn changes the values of the detection parameters. Thus, a change or period of high fluctuation over time in the detection parameters measured between two electrodes may be indicative of movement of the body.

For example, two electrodes in the electrode array 104 may be located on the front upper and lower sides of the user's knee, respectively. When the user's leg is straight, a detection pulse between the pair may pass primarily across the user's skin without significantly penetrating into the tissue. As the user's leg bends, the primary electrical pathway taken by detection pulses may pass more through the muscle or nerves of the user's leg. A detection pulse sent between the electrode pair when the leg is straight would measure different detection parameter values compared to a pulse through a bent leg. Thus, movement of the knee can be detected. In other examples, the movement of the body to which the electrodes are physically attached may not change the pathway which the detection pulse travels along but the contact resistance of the electrodes with the skin may vary as the body moves relatively to the electrodes.

Using a detection mode that utilises electrodes of the electrode array 104 provides several advantages. Firstly, the ES system 102 can be used to provide sensitive and high-resolution movement detection. It has been found that even small movements and muscle twitches produce changes that are detectible by the electrodes applying detection pulses. By generating electrical parameter detection bursts between a plurality of different combinations of electrodes, the controller 101 can collect enough data to precisely locate and identify a user movement. Additionally, the same electrode array 104 can be used for stimulation and movement detection, meaning that there is no need for an expensive additional set of sensors which must be integrated around each other.

The detection mode can also be used to check whether the electrode array has been correctly positioned on the user's body. When the electrodes are not positioned close to the user's skin, there will be no electrical path between the electrodes and the resistance calculated between any pair of electrodes will be infinite. Thus, if infinite resistance is calculated for every pair of electrodes in an electrode array, it is likely that the array is not positioned against a user. Similarly, if infinite resistance is calculated for electrode pairs including electrodes in a particular region of the electrode array, it is likely that that region of the electrode array is not correctly positioned against the user's body.

The detection period is a period of time in which detection of one or more detection parameters indicative of a degree of movement of the muscle can be conducted. Generally, detection periods do not overlap with the application of a stimulation pulse, because the lower-intensity detection pulses can be masked by higher-intensity stimulation pulses between the same or neighbouring electrodes. However, in some embodiments, detection may occur between one subset of electrodes in the electrode array 104 while another subset of electrodes is used for stimulation. The detection period is also distinct from a low-power period, in which the controller 101 and/or ES system 102 may be configured into a standby mode. During the detection period the controller 101 may be configured to receive and record detection parameters from the electrode array 104 in order to calculate the electrical parameters for each electrode pair. Detection parameters and electrical parameters are discussed further below. The values of the electrical parameters may be calculated concurrently during the detection period, or alternatively to reduce computational load the detection parameters may be recorded and the electrical parameter values calculated subsequent to the detection period.

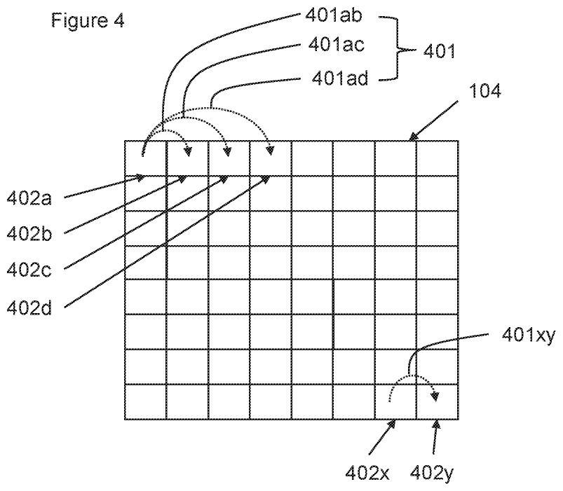

Referring to FIG. 4, the signalling provided by the controller 101 during the detection mode causes the ES system to provide a plurality of electrical parameter detection bursts 401 between pairs of electrodes 402. Each electrical parameter detection burst 401 comprises one or more detection pulses. The electrical parameter detection bursts 401 may be provided sequentially, such that an electrical parameter detection burst 401ab between electrodes 402a and 402b is provided first, followed by electrical parameter detection burst 401ac between electrodes 402a and 402c, which is then followed by electrical parameter detection burst 401ad between electrodes 402a and 402d. The controller 101 may also provide electrical parameter detection bursts 401 concurrently. This reduces the time required to run electrical parameter detection bursts between all possible pairs of electrodes. However, care must be taken that the electrical discharges from concurrent electrical parameter detection bursts 401 do not interfere with each other, as this could distort the detection parameter measurements. Thus, if an electrical parameter detection burst 401 is provided between a first pair of electrodes both located in one region of the array 104, a second electrical parameter detection burst 401 should only be provided concurrently between two electrodes that are both located distally from that region. For example, electrical parameter detection burst 401ab between electrodes 402a and 402b can be provided concurrently with electrical parameter detection burst 401xy between electrodes 402x and 402y, because electrodes 402a and 402b are both located distally to electrodes 402x and 402y. However, electrical parameter detection burst 401ab should not be provided concurrently with electrical parameter detection burst 401ac between electrodes 402a and 402c, because electrodes 402a and 402b, and 402c are located close together.

In addition to the detection pulses, an electrical parameter detection burst 401 may include discrete intervals of time for recording measurements of the detection parameters, or for performing calculations.

Multiple electrical parameter detection bursts 401 may be provided between a single pair of electrodes during a detection period. This allows changes to the detection parameters (and thereby movement) to be detected from a single detection period. However, electrical parameter detection bursts 401 will always be provided between at least two different pairs of electrodes of the electrode array during a single detection period.

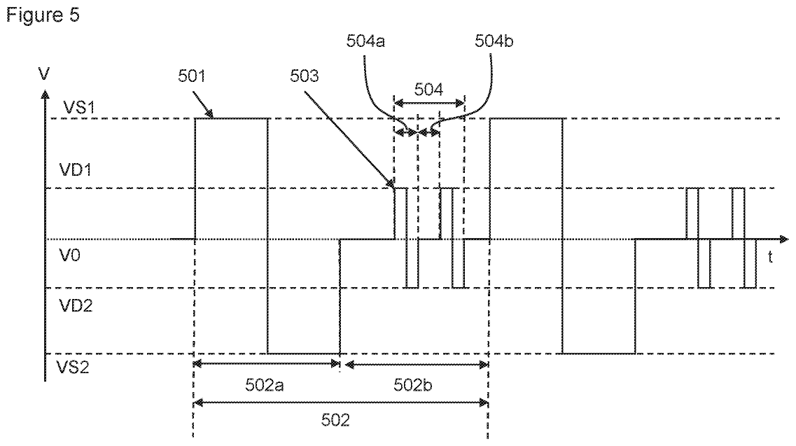

FIG. 5 is a graph showing the relative voltage applied between a pair of electrodes where time is plotted along the x-axis and relative voltage is plotted along the y-axis. In this example, the electrode array 104 is first configured to selectively operate in a stimulation mode based on signalling received by the ES system 102 from the controller 101. In the ES system 102, electrical stimulation applied during the stimulation mode may be provided a plurality of times in succession to the user in order to provide for muscle contraction and increased blood flow.

Each stimulation pulse 501 can be applied over a stimulation duration 502a. The stimulation pulses 501 may be applied consecutively and in an uninterrupted manner such that, as a first stimulation pulse is completed, a subsequent second stimulation pulse begins immediately. In other examples, one or more or each stimulation pulse may be separated by a rest period 502b during which no stimulation pulses 501 are applied. While operating in the stimulation mode, a single stimulation cycle 502 can be defined from the start of a first stimulation pulse to the start of a second stimulation pulse that follows the first stimulation pulse 501. In examples where there are no rest periods 502b between stimulation pulses 501, the duration of the stimulation cycle 502 is equal to the stimulation duration. In examples where rest periods 502b are provided between successive stimulation durations 502a, the duration of the stimulation cycle is equal to the stimulation duration 502a plus the duration of the rest period 502b.

The application of a stimulation 501 pulse comprises applying a stimulating potential difference applied between a first stimulation electrode and a second stimulation electrode. Each stimulation pulse 501 may be separated by a rest period 502b which is a period of time which is absent of stimulation pulses. Thus, each stimulation pulse 501 may be described as the application of a stimulation signal wherein each stimulation signal comprises the application of a first stimulation voltage to the first stimulation electrode and a second stimulation voltage to the second stimulation electrode to provide the electrical stimulation to the muscle or its innervation via the skin between the first and second stimulation electrodes. The first stimulation voltage is different to the second stimulation voltage in order to provide for the stimulating potential difference. It will be appreciated that the number of stimulations applied to the muscle will vary depending on the user and on the desired effect.

The controller 101 is configured to provide signalling to the ES system 102 such that, the stimulation signal is applied as a monophasic or a biphasic waveform. A biphasic waveform can be produced by applying a voltage VS1 at the first electrode, while the second electrode is held at a reference voltage V0, which may be a ground voltage. Following the application of the VS1 and V0 voltages, a voltage VS2 is applied at the second electrode, while the first electrode is held at the reference voltage V0. The magnitudes of the first and second stimulation voltages VS1 and VS2 may be the same as each other or they may be different. The controller 101 may be configured to provide signalling to the ES system 102 such that the first stimulation voltage VS1 and the second stimulation voltage VS2 are applied as square waves of opposing polarities. This may provide for particularly stable and consistent electrical stimulation. Alternatively, the stimulation voltages that provide for a stimulation potential difference between electrodes may be provided as sinusoidal AC voltages wherein each new application of a stimulation signal corresponds to the sinusoid passing through the reference voltage which may be at a relative 0 volts.

The controller 101 may be configured to provide for the application of the stimulation signals at a fixed rate, such that the stimulation pulses 501 have a consistent pulse frequency and pulse duration. For example, a rate of stimulation signal application may be between 1-100 Hz. In particular the rate of stimulation signal application may be 30-40 Hz and, more particularly, the rate of stimulation signal application may be 36 Hz. As described above, each stimulation pulse 501 has a stimulation duration 502a and may be separated from adjacent stimulation pulses 501 by rest periods 502b. The stimulation durations 502a are determined by the pulse frequency of the stimulation pulses 501 and the duration of the rest periods 502b used. It will be appreciated that the pulse frequency (the number of stimulation pulses per second) will be effectively equal to the number of stimulation cycle 502 frequency (the number of stimulation cycles 502 per second). For example, if the stimulation pulse frequency is 36 Hz and the stimulation signals are separated by rest periods of 27.7 ms, then the stimulation duration 502a will be 0.175 ms. One may refer to the proportion of the stimulation cycle over which the stimulation pulses 501 are applied as the duty cycle of the signal. In this way, where the stimulation signal is applied over half of the stimulation cycle, then the stimulation mode may be operating at a 50% duty cycle.

The controller 101 may further be configured to provide for the control of one or more other parameters related to the application of stimulation signals 501 or the electrical parameter detection bursts. For example, the controller 101 may provide control of the phase-duration and inter-phase duration. The controller 101 may provide for control of the ramp-up time and the ramp-down time where these times correspond to the amount of time taken to get to the desired first and second voltages and down from the first and second voltages, respectively. The controller may also provide for control of a plateau time when neither ramp-up time nor ramp-down time is being enacted.

The ES system may operate in a detection mode during a rest period 502b, such that a detection period can be defined as the period over which a plurality of electrical parameter detection bursts 503 are applied between pairs of detection electrodes of the electrode array. The detection electrodes may be the same electrodes as the stimulation electrodes or may be different electrodes. The minimum duration of the detection period may be determined by the speed of the processor used by the controller and the number of electrodes in the electrode array 104 to be used for detection. The relationship of the detection period to the processor speed of the controller exists because no therapeutic or other effect is desired when an electrical parameter detection burst is applied to the user. Instead, one only needs to take an electrical parameter measurement, such as a resistance measurement between the two detection electrodes. In one example, the minimum duration of a detection period expressed in seconds would be given by (1/(processor frequency/2))×((N(N−1))/2) where N is the number of detection electrodes in the electrode array 104. If an electrode array 104 has 64 electrodes used for detection and the processor frequency is 480 MHz, then a detection period of 8.4 microseconds will be needed to pass detection pulses between all possible pairs of electrodes. The maximum duration of the detection period may be the duration of the rest period 502b, so that the detection periods can be interspersed with the stimulation pulses 501.

Each electrical parameter detection burst 504 comprises one or more detection pulses 503. The example shown in FIG. 5 shows only a single detection pulse 503 per electrical parameter detection burst 504. In other examples, each electrical parameter detection burst 504 may comprise a plurality of detection pulses 503 such that each electrical parameter detection burst is a wave packet of detection pulses 503. The detection pulse duration is generally determined by the processing speed of the controller 101, with the pulse duration 504a being the shortest possible time over which the controller can generate the detection pulse 503. The electrical parameter detection bursts 504 may be separated by inter-burst intervals 504b. For example, if the controller 101 is capable of generating an electrical parameter detection burst 503 comprising a single detection pulse with a 1 ms pulse duration, then, in the absence of inter-burst intervals, the pulse frequency will be 1000 Hz. The detection pulses may be provided at a lower frequency but with the same duration per pulse by providing extended inter-burst intervals between the electrical parameter detection bursts or by providing inter-pulse intervals between pulses of a single electrical parameter detection burst. For example, two electrical parameter detection bursts each comprising single detection pulses with 1 ms durations may be generated with an inter-burst interval having a duration of 98 ms. In general, the detection pulse frequency may be between 10 Hz and 100 MHz.

As with the stimulation pulses 501, the detection pulses may consist of a biphasic square wave with a maximum potential difference VD1 and a minimum potential difference VD2. Each electrical parameter detection burst 504 is configured to not provide for muscle stimulation (activation of the muscle such that the muscle fibres “twitch”). That is, each electrical parameter detection burst is configured to have at least one or more of a signal amplitude, frequency, and/or waveform set at a level such that the detection burst is insufficient to cause muscle activation-inducting stimulation at the target body location. In particular, at least one or more of the signal amplitude, frequency and waveform may be selected such that they are insufficient to cause muscle stimulation at the target body location. That is, stimulation is avoided by the selection of appropriate waveform parameters. In particular, the electrical parameter bursts are configured such that they would not provide for muscle-activation inducing stimulation of a muscle at the target location when provided in isolation. In FIG. 5, the absolute potential difference |VD1−VD2| of a detection pulse 503 is less than the absolute potential difference |VS1−VS2| of a stimulation pulse 501, such that the detection pulses 503 do not cause muscle activation-inducing stimulation. For example, one parameter to avoid muscular stimulation may be to maintain the current supplied between each pair of detection electrodes during each detection pulse 503 at no more than 0.1 mA.

In alternate examples, a calibration may be performed for each patient to determine the intensity of the stimulation 501 and detection pulses 503. For example, starting from a low intensity (an intensity at or below a minimum expected intensity for causing stimulation), pulses of increasingly higher intensity may be applied to the user until muscle activation is observed at a certain threshold intensity. The stimulation pulses 501 are then set to have an intensity at or above this threshold, while the detection pulses 503 are set to have an intensity below the threshold.

It may be that the electrical parameter detection bursts 504 are provided intentionally simultaneously with another signal, such as with a stimulation signal, and that the other signal is capable, either alone or in combination with the electrical parameter detection burst, to provide for stimulation of a nerve or muscle at the target body location. Such an embodiment would still fall within the scope of the present disclosure. In other examples, the electrical parameter detection bursts 504 may be provided in isolation to the stimulation pulses 501.

Interspersing stimulation pulses 501 and electrical parameter detection bursts 504 allows changes in electrical parameter values in response to stimulation to be calculated. As shown for clarity in FIG. 5, there may be a delay between the end of a stimulation pulse 501 and the beginning of a detection period. Alternatively, the detection period may begin immediately after the end of a stimulation pulse. In one or more embodiments, the electrical parameters may be calculated a plurality of times during a detection period in order to monitor a change in the electrical parameters over the course of a detection period. For example, an electrical parameter may be calculated from a first electrical parameter detection burst 503, and then re-calculated from a subsequent electrical parameter detection burst. This allows changes in the electrical parameters between a pair of electrodes to be detected.

The stimulation pulses 501 and individual detection pulses 503 of electrical parameter detection bursts 504 as shown are both biphasic square waves. However alternatively, either or both the stimulation and detection pulses may be monophasic. In other examples, the stimulation and detection pulses may comprise either monophasic or biphasic sinusoidal waveforms, sawtooth waveforms, or another waveform shape.

The controller 101 is further configured to detect one or more detection parameters indicative of the degree of movement of the body associated with each electrical parameter detection burst. That is, the controller may receive signalling from the detection electrodes indicative of at least one of an electrical resistivity, an electrical resistance, an electrical conductivity, an electrical conductance, an electrical impedance, and an electrical admittance. The listed electrical parameters may be indicative of the degree of movement of the body to the detection pulse because any of these parameters will provide an indication of the charge transferred through the body. For example, the electrical resistance of charge transferred through the body (the current through the body) during a detection pulse is dependent on several factors which include at least the transcutaneous, transsubcutaneous, or transmuscular resistance and the contact resistance of the electrodes with the body. By obtaining a measurement of the electrical resistance, one obtains an indication of changes in the contact resistance and, in this way, it is possible to tell if the muscle is, or has been, moving. In this example, the detection parameter may be the current that passes through the nerve, muscle, or any other tissue located in the electrical pathway between the detection electrodes. The provision of the signalling, which may be referred to as measurement signalling, may be performed simply by providing a voltage to the controller 101 indicative of a measured value if the electrode array 104 is in electrical contact with the controller 101. In other embodiments, the measurement signalling may comprise a wireless signal, for example, transmitted by an ES system controller to the controller 101.

The controller 101 is further configured to calculate the electrical parameter value between each pair of detection electrodes based on the one or more detection parameter values. For example, the controller may be configured to calculate a resistance (electrical parameter) between the two detection electrodes based on the current (detection parameter) that flowed through the muscle, nerve or any other tissue located in the electrical pathway between the detecting electrodes as a result of the potential difference being applied between the two electrodes of the pair of detection electrodes,

As defined herein, a pair of electrodes refers to the combination of a first detection electrode and a second detection electrode. At least one subsequent electrical parameter detection burst is not provided between the same two detection electrodes in combination during a given detection period and is, instead, provided between a different combination of two electrodes in the array. For example, if electrode A and electrode B have a potential difference applied thereacross for the provision of a first electrical parameter detection burst, then at least one subsequent electrical parameter detection burst in the plurality of electrical parameter detection bursts will not use electrodes A and B during the same given detection period and, instead, they will use electrodes A and C, B and C or electrodes C and D during the same given detection period. This description does not entirely limit repeat measurements being performed, however, there will be a plurality of measurements performed on different pairs of electrodes in order to provide for electrical parameter detection bursts that effectively determine electrical parameters for multiple pairs of electrodes.

In one or more embodiments the electrode array 104 may be caused to operate in a detection mode such that all combinations of pairs of electrodes in the electrode array 104 have potential differences applied across them in the form of individual measurements in the course of a single detection period. The controller may be configured to iterate through all possible pairs of electrodes by selecting a first electrode and then sending electrical parameter detection bursts between that electrode and every other electrode in the array 104 sequentially. The controller then selects a second electrode, and sends electrical parameter detection bursts between the second electrode and every other electrode in the array 104 save for the first electrode. This process is repeated until electrical parameter detection bursts have been passed between all possible pairs of electrodes in the array. In other examples, the order in which the pairs of electrodes are selected may be according to a different ordering or the order may be randomised.

By providing for measurements between each definable electrode pair in an electrode array, the maximum possible number of sensors during the detection mode may be provided, thereby providing the greatest possible measurement resolution available through this technique. In one or more embodiments, the controller 101 may be configured to provide signalling to the ES system to cause the ES system to provide an electrical parameter detection burst between at least 50% of the pairs of detection electrodes that can be defined within the electrode array and correspondingly detect one or more detection parameters indicative of the electrical parameter which is, in itself, indicative of the degree of movement of the body. Alternatively, the percentage of activated possible pairs of electrodes may be at least 30%, at least 40%, at least 60%, at least 70%, at least 80% or at least 90%. By taking a large number of measurements between different pairs of electrodes that can be defined within the array, one effectively creates a corresponding number of sensors which can provide improved accuracy, reliability and location specificity compared to, instead, using a small handful of flex sensors, for example.

The maximum resolution in the detection mode can be achieved by providing electrical parameter detection bursts between all possible pairs of electrodes. For an electrode array 104 containing N total electrodes, the number of possible pairs is given by the binomial coefficient C(N,2), which is equal to N!/(2!(N−2)!)=(N−1)(N/2). For example, in a 2×2 array, there are 6 unique pairs of electrodes that can be defined. The number of combinations of electrode pairs increases rapidly as the number of electrodes in the electrode array 104 increases. For example, there are 45 possible electrode pairs in an electrode array 104 with 10 electrodes, 4950 possible electrode pairs in an electrode array 104 with 100 electrodes, and 11175 possible electrode pairs in an electrode array 104 with 150 electrodes. Therefore, in electrode arrays 104 larger than a certain size it may become impractical to provide electrical parameter detection bursts between all possible pairs of electrodes. Instead, electrical parameter detection bursts may be sent between groups of electrodes, or between a subset of the possible pairs.

The electrode array 104 may be divided into a first sub array and a second sub array, each sub array corresponding to a different portion of a muscle that the electrodes of the electrode array are expected to contact. In these embodiments, each electrode of the first sub-array form a pair of electrodes with each electrode of the second sub-array such that each electrode pair comprises one electrode from each sub-array. This may provide for the avoidance of testing electrode pairs which would be expected to be associated with the same motor point 106, thereby providing for sensors that are spaced further apart from each other which may obtain more reliable measurements of movement of a muscle, for example.

The provision of signalling from the controller 101 to cause the ES system to operate in the detection mode provides for a system which is able to effectively create a large number of sensors distributed all across the body to which the electrode array is affixed without needing to add extra components that take up both space and power. This allows for the provision of an ES system that is more compact and more comfortable for a user which, in turn, leads to consistently improved compliance by the user.

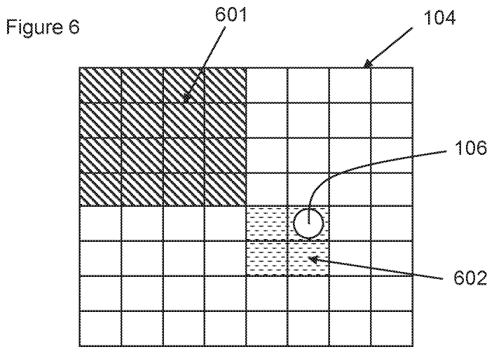

FIG. 6 shows an example electrode array 104 defining a 4×4 group-electrode 601, a 2×2 group electrode 602 and a motor point 106. The electrode array 104 may be configured such that a plurality of individual electrodes can have voltages applied thereto contemporaneously to define a larger group-electrode 601, 602. The voltages applied to the group electrode 601, 602 may be substantially identical. The electrical contiguity of the electrodes allows for the simulation of a single large electrode and may define the electrodes in any shape suitable for the stimulation of a motor-point. Such group-electrodes may be used as one or both of stimulation electrodes and detection electrodes. In one or more examples, the group-electrodes may be substantially spot-like as opposed to defining a line of electrodes. For example, the group-electrodes 601, 602 may be defined by a substantially square or circular grid, such as the 4×4 grid of electrodes 601 in a regular matrix electrode array 104. One or more group-electrodes 601, 602 may be used for improving the efficiency of detecting the response of a muscle or nerve to electrical stimulation or they may be used to provide for stimulation of larger areas on the body during a stimulation mode. Alternatively, or additionally, the one or more group-electrodes 601, 602 may be used for detecting a degree of movement of a muscle associated with the group-electrodes without the need for stimulation to be instigated. In the case of certain muscle groups, a group-electrode may provide for improved muscle stimulation to provide for stimulation of a single motor point 106 using a plurality of individual electrodes.

Using group-electrodes for detection offers a number of advantages. Searching through all combinations of electrodes in the array 104 is time consuming, and becomes increasingly more difficult as the number of electrodes in the array 104 increases. Using group-electrodes may significantly decrease the total measurement and processing time while providing sufficiently high resolution to detect muscle movement resulting from muscular stimulation.

Group-electrode detection may also be used as part of algorithmic search patterns to more efficiently detect and locate movement. For example, the electrode array 104 may be divided into large scale group-electrodes. An initial set of electrical parameter detection bursts are provided between the group electrodes, and the calculated electrical parameters are used to give an indication of the movement level associated with each group-electrode. The group-electrodes with high movement levels are divided into smaller group-electrodes, and electrical parameter detection bursts are provided between the smaller group-electrodes. This process continues until the controller has located the individual electrode or small group-electrode associated with high movement levels, thereby identifying the location of the movement. This approach may be more efficient than simply sending electrical parameter detection bursts between every possible combination of electrode pairs.

Instead of providing for stimulation of motor points 106 using single electrodes during the stimulation mode, some muscles may be stimulated better by providing for stimulation over an area greater than that of a single electrode. As such, the controller 101 may be configured to identify a first stimulation group-electrode and a second stimulation group-electrode where each group-electrode is aligned with a different motor point 106 of the muscle of the user, The controller 101 may then be configured to provide signalling to cause the ES system 102 to operate the electrode array 104 in a group-electrode stimulation mode wherein the signalling causes a plurality of stimulation signals to be applied first and second stimulation group-electrodes. Each stimulation signal may comprise an application of a first stimulation voltage to the first stimulation group-electrode and a second stimulation voltage to the second stimulation group-electrode to provide electrical stimulation to the muscle or its innervation via the skin between the electrode pair and wherein the first stimulation voltage has a different polarity to the second stimulation voltage. It will be appreciated that the group-electrode stimulation mode is directly analogous to the stimulation mode previously described except with effectively larger electrodes. Analogously, a group-electrode detection mode may be provided.

In order to prevent problems caused by inactivity of a user, the controller 101 may be configured to detect inactivity of the user and, if the user has been inactive for a predetermined period of time, the controller 101 may be configured to initiate at least a stimulation mode. In particular, the controller 101 may be configured to cause the ES system 102 to operate the detection mode even when the ES system has not operated in the stimulation mode recently. This detection mode may be referred to as a passive detection mode, however, it operates in the same way as the detection mode already described (which may be referred to as an active detection mode). During the detection mode, the controller 101 is configured to not provide signalling to cause the ES system 102 to apply voltages at any of the electrodes at a sufficient level to cause stimulation of one or both of a muscle and a nerve. During the detection mode, the controller 101 is further configured to receive signalling from the ES system 102 indicative of the detection parameters one of continuously and periodically. It will be appreciated that either continuous or periodic monitoring of the activity of the user can provide for valuable monitoring of the activity of the user. The ES system 102 may operate in this detection mode by default when not operating in another mode.

User-induced stimulation refers to a contraction of the muscle caused by the user purposefully using the muscle in question. A movement may only be considered to be a user-induced stimulation if the controller 101 determines, based on signalling received during a detection period, that the contraction is within a predetermined threshold of a calibration signal. The calibration signalling is signalling indicative of the contraction of the muscle by the user during a calibration mode. That is, small amounts of movement by a user (below a muscle reaction threshold or a threshold of the calibration signalling) may not be sufficiently stimulating to provide for desired blood flow or exercise of the muscle. In such circumstances, it may be desirable to have the ES system 102 operate in the stimulation mode.

If the controller 101 receives no indication of user-induced stimulation within a detection period of the passive detection mode, the controller 101 may be configured to cause the ES system 102 to operate in an alternate mode, such as in the stimulation mode. Other alternate modes may include a calibration mode for determining baseline electrical parameter values or a motor-point scan mode configured to identify preferred motor points for stimulation. The detection period during the passive detection mode may be, for example, 30 minutes, one hour, two hours, three hours or any other suitable time based on the needs of the user. The controller 101 may further be configured to cause the ES system 102 to operate in the detection mode during or after the operation of the system in the stimulation mode.

In some examples, registered contraction of the muscle that is below the predetermined threshold of the calibration signalling over a predetermined upper contraction duration may be registered as a user-induced stimulation, as this may be indicative of small-scale exercise over an extended period that is sufficient to obviate the need of additional stimulation by the electrode array 104. That is, movement by the user by small amounts (below a threshold) for an extended period of time may be deemed to be acceptable to cause the desired blood flow, exercise or other stimulation effect for the user such that stimulation by the ES system 102 is deemed to be unnecessary.

The controller may be configured to generate an electrical parameter map representative of a spatial distribution of the electrodes of the electrode array, wherein the electrical parameter value map comprises a plurality of elements arranged in a grid and wherein each element comprises an element value based on one or more electrical parameters calculated by the controller 101. That is, the electrical parameter map is a two-dimensional numerical representation of the two-dimensional face of the electrode array based on electrical parameters calculated between pairs of electrodes. The grid layout of the electrical parameter map facilitates detection of patterns in the values of the elements, allowing particular kinds and locations of movement to be identified. The electrical parameter map may be easily stored as a matrix or array on a computer-readable storage medium, so that instances of the electrical parameter map calculated from different electrical parameter detection bursts can be compared against each other.

In one embodiment, each element of the electrical parameter map corresponds to an electrode of the electrode array, and the element value of an element of the electrical parameter map is based on a sum or average of each calculated electrical parameter that used the corresponding electrode. For example, the value of the element in the first row, first column of the electrical parameter map may correspond to the electrode in the first row, first column of the electrode array 104. The value of that element may then be set as the sum of the resistance values (resistance being an exemplary electrical parameter) calculated between that electrode, and every other electrode in the electrode array 104.

It will be understood that there may not be a one-to-one correlation between each element of the electrical parameter map and each electrode of the electrode array. For example, to reduce the size of the electrical parameter map or increase robustness, each element of the electrical parameter map may correspond to a cluster of electrodes. In this way, a 4×4 electrode array 104 could be represented by a 2×2 electrical parameter map. Conversely in yet other embodiments, the spatial extent represented by each element may be smaller than a single electrode and, as such, the spatial extent of each electrode may be represented by a plurality of elements.

An electrical parameter map as described above allows the location of particular movements to be easily identified. For example, if a user's movement causes an electrode to change position slightly relative to the user's skin, then the paths between that electrode and every other electrode will change over time. The resistance between this electrode and other electrodes will change more than the resistance between pairs of electrodes which have not changed position. Hence, a larger change in the value of an element of the electrical parameter map may indicate that there has been a localised user movement near the electrode which is represented by that element. Similarly, a large change in the value of a plurality of elements representing electrodes in a particular region of the electrode array 104 may indicate user movement throughout the area covered by that region of the electrode array.

In order to track changes in element values stored in the elements of the electrical parameter map, the controller 101 may be configured to calculate an electrical parameter map from electrical parameters measured during a current detection period, wherein the element values of the electrical parameter map are based on a difference between element values calculated during the current detection period, and element values calculated during a preceding detection period taken at a time earlier than the current detection period. In other words, each element of the electrical parameter map represents the difference or delta between the electrical parameters as measured over a time interval. A larger absolute value in any element compared to the preceding detection period therefore indicates user movement around the electrodes which that element represents. The preceding detection period may be a most recent detection period taken as compared to the current detection period. That is, the preceding detection period may be the detection period preceding the current detection period which may: immediately precede the current detection period; be separated from the current detection period by a time delay; or be separated from the current detection period by one or more stimulation durations during which stimulation pulses are applied to the user.

Rather than comparing the electrical parameter values from a current detection period to those of a preceding detection period, the electrical parameter values can be compared against predefined element values. Predefined baseline electrical parameters may, for example, be electrical parameters obtained during a calibration measurement taken at a point in the past during a calibration mode. Alternatively, the baseline electrical parameters may be standardised values that are not based on measurements taken by the electrical stimulation system, with comparison against these values used to detect abnormal deviations.

The controller 101 may generate an electrical parameter map in which each element of the electrical parameter map corresponds to a pair of electrodes of the electrode array. The value of each element of the electrical parameter map may be the value of the electrical parameter calculated for the corresponding pair of electrodes. As described above, the element value of an element of the electrical parameter may be based on the difference between an electrical parameter calculated between the pair during the electrical parameter detection burst, and an electrical parameter calculated between the pair during a previous electrical parameter detection burst. An element of the electrical parameter map may correspond to a plurality of pairs, and the value of that element calculated as the sum or average of the electrical parameter between each of the corresponding pairs. This allows different kinds of movements to be discerned. For example, one element of the electrical parameter map may correspond to pairs of electrodes separated by a short distance, while another corresponds to pairs of electrodes which are more distantly separated. Movement which causes changes in transmuscular resistance, but not transcutaneous resistance (such as small muscle twitches), would cause more change to the value of the second element than to the first element.

The controller 101 may be configured to determine, based on the electrical parameter map, whether to provide signalling to the ES system to operate the electrode array in the detection mode or in an alternate mode such as the stimulation mode. It will be appreciated that the values in the electrical parameter map will be indicative of a change in the electrical parameter over time and over the spatial distribution represented by the electrical parameter map. Movement of the body location to which the electrodes are coupled will result in changes in the element values of the electrical parameter map. As such, the controller may use the element values in the electrical parameter map as indicators of whether a user of the system has been physically active over a particular time period, such as the time period between the preceding detection period and the current detection period. As indicated above, the element values may be defined in any of numerous different ways such as: a difference between a previous value and a current value or predefined value; a ratio between a previous value and a current value or predefined value; a sum of a previous value and a current value or predefined value; or any number of other ways of defining the element values. As such, there are correspondingly numerous ways to define in what situation the element value is indicative of non-movement of the user. For example, the controller may be configured to provide signalling to the electrical stimulation system to operate the electrode array in the detection mode or in a stimulation mode based on one or more of the element values being above a predetermined threshold or below a predetermined threshold. Alternatively, the controller may be configured to detect if the change in the element values is greater or less than a predetermined threshold change. In yet other examples, the controller 101 may be configured to provide signalling to the ES system 102 to operate the electrode array 104 in the detection mode or in the stimulation mode if a predetermined number of consecutive element value changes are greater or less than a predetermined threshold.

It has been found that the resistance and impedance of skin decreases as the frequency of the applied electrical signal increases. If the stimulation pulses are applied at a comparatively low frequency (in the 10s of Hz), then skin resistance will be high (in the 100s to 1000s of Ohms). This high resistance causes high localised energy dissipation, leading to increased discomfort for the user as well as higher energy requirements for the ES system 102. This can be mitigated by applying skin resistance reduction pulses while operating in the stimulation mode, wherein the skin resistance reduction pulses are applied at a higher frequency and lower intensity than the stimulation pulses. While not wishing to be bound by theory, it is believed that the skin resistance reduction pulses re-align the ions in the user's skin, increasing the conductivity of the skin and reducing energy dissipation.

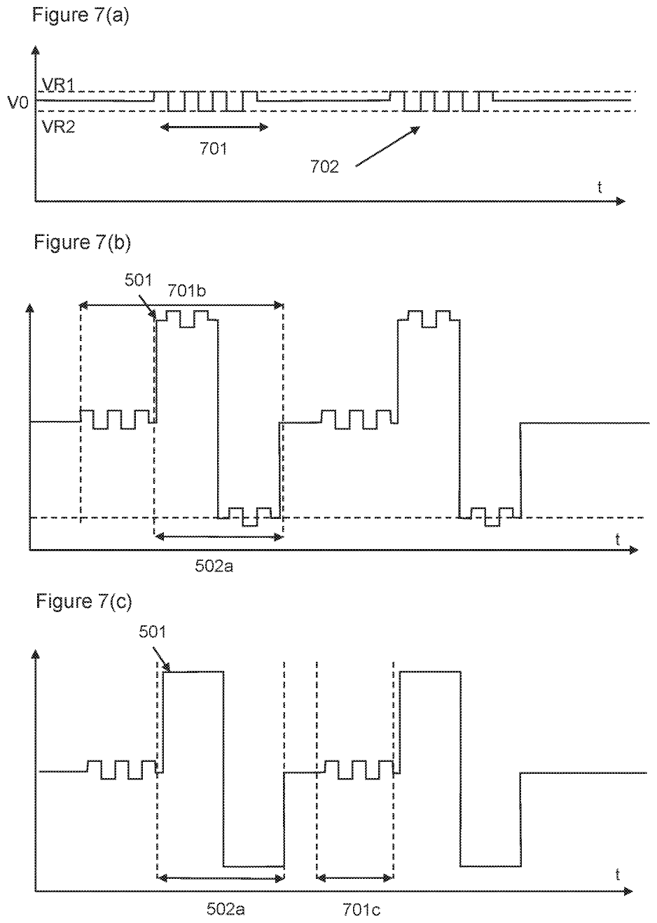

Turning to FIG. 7, the controller may be configured to, while operating in the stimulation mode, cause the ES system to apply a plurality of skin resistance reduction pulses 702. FIG. 7(a) shows a representation of the skin resistance reduction pulses 702 in isolation plotted as voltage against time, without any stimulation pulses. The skin resistance reduction pulses 702 are generated by generating an additional potential difference VR1 at one stimulation electrode, relative to the other stimulation electrode's voltage V0, then holding the first electrode at V0 while the other electrode is held at voltage VR2. The skin resistance reduction pulses 702 are lower-intensity than the stimulation pulses (i.e. the skin reduction pulses 702 have a voltage amplitude |VR2-VR1|lower than the voltage amplitude of the stimulation pulses), but are applied at a higher frequency than the stimulation pulses. The skin resistance reduction pulses 702 are applied in skin resistance reduction bursts 701. The skin resistance reduction pulses 702 are biphasic square waveforms, but may alternatively be monophasic or biphasic sinusoidal waveforms, sawtooth waveforms, or another waveform shape.

As shown in FIG. 7(b), the skin resistance reduction pulses 702 can be superimposed onto stimulation pulses 501. As will be appreciated, this will result in constructive interference that provides combined waveforms that comprise a high frequency repeating waveform on the lower frequency and higher magnitude waveform. The skin resistance reduction burst 701b overlaps and precedes the stimulation duration 502a of the stimulation pulse 501 in its entirety. Rather than having bursts 701b, the train of skin resistance reduction pulses 702 may be continuous while the ES system is operating in the stimulation mode.

In one or more embodiments, the skin resistance reduction pulses 702 may carry a current no more than 0.1 mA per pulse, and have a frequency between 1 and 100 000 Hz. This ensures that the skin resistance reduction pulses are high-frequency enough to significantly decrease skin resistance for the stimulation pulses 501, without significantly affecting the intensity of the stimulation pulses 501.

As shown in FIG. 7(c), rather than superimposing the skin resistance reduction pulses 702 to overlap with the stimulation duration 502a of the stimulation pulses 501, the controller may alternatively be configured to synchronise the skin resistance reduction pulses 702 so that they do not overlap with the stimulation pulses 501. That is, the skin resistance reduction bursts 701c are applied in the intervals between stimulation pulses 501.

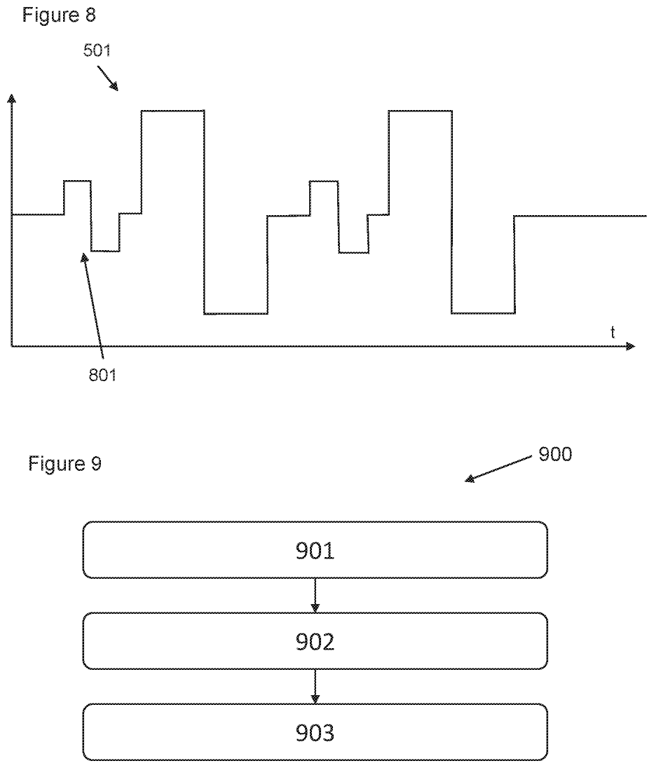

FIG. 8 shows sensory blocking pulses 801, synchronised with the stimulation pulses 501 in the same manner as the skin resistance reduction pulses 702 were in FIG. 7(b). The sensory blocking pulses 801 may also be superimposed with either or both the stimulation pulses 501 or the skin resistance reduction pulses 702.

The sensory blocking pulses 801 cause the user to experience less discomfort as stimulation pulses 501 are applied. When the stimulation pulses 501 are applied, high-intensity signals may be generated in the user's sensory nerves. These then travel via the spine to the brain where they are experienced as pain. The sensory blocking pulses 801 cause low-intensity nerve signals to be generated instead, blocking the signals transmitted via the sensory nerves at the spinal level from transmitting high intensity pain signals to the brain when the stimulation pulses 501 are applied. In this sense, sensory blocking pulses 801 work in a similar fashion to rubbing a region of the body after a minor injury such as a bruise or scrape. The rubbing sensation “blocks” out the pain from the injury, and the individual feels less pain as a result. The sensory blocking pulses 801 may be applied through a plurality of electrode pairs surrounding the pair of electrodes being used for stimulation. For example, the sensory blocking pulses 801 may be applied through a group electrode surrounding the stimulation electrodes, generating low-intensity blocking signals over a wide area of skin that block out the pain signals.

The sensory blocking pulses 801 are generally less intense than the stimulation pulses 501, but are generally higher intensity and lower frequency than the skin resistance reduction pulses. In one example, the sensory blocking pulses 801 are applied at a pulse frequency between 0.1 and 150 Hz. In an example, the sensory blocking pulses 801 carry a current between 1-10 mA lower than that carried by the stimulation pulses 501. In another example, the current carried by the sensory blocking pulses 801 is calibrated for a user. The sensory blocking pulses 801 are initially applied to the user at a current of 0.1 mA or higher, and then the current is increased until the user begins to experience pain relief due to the sensory blocking effect.

With reference to FIG. 9, a method 900 for providing ES to a muscle of a user using the previously described system 100 will now be described. The method comprises steps of, by way of the controller, providing 901 signalling to the ES system to operate the electrode array in a detection mode during a detection period, the signalling causing the ES system to provide a plurality of electrical parameter detection bursts during at least one detection period wherein each electrical parameter detection burst comprises one or more detection pulses and wherein each electrical parameter detection burst is configured to not provide for muscular stimulation. The method further comprises detecting 902, by way of the controller, one or more detection parameters indicative of the degree of movement of the body associated with each electrical parameter detection burst. Each electrical parameter detection burst is provided by applying a potential difference between different pairs of the plurality of electrodes. A pair of electrodes refers to the combination of a first detection electrode and a second detection electrode selected from any electrode of the electrode array such that, when the controller provides a subsequent electrical parameter detection burst, the corresponding electrode pair for that subsequent electrical parameter detection burst comprises a pair of any two electrodes of the electrode array except for any electrode pair which would correspond to a previously provided electrical parameter detection burst of the detection mode. It will be appreciated that the discussion of avoiding using the same two electrodes of the electrode array refers to the repeated use during a given rest period or a single implementation of operation in the detection mode. The method yet further comprises, by way of the controller, calculating 903 the electrical parameter between each pair of detection electrodes based on the one or more detection parameters.

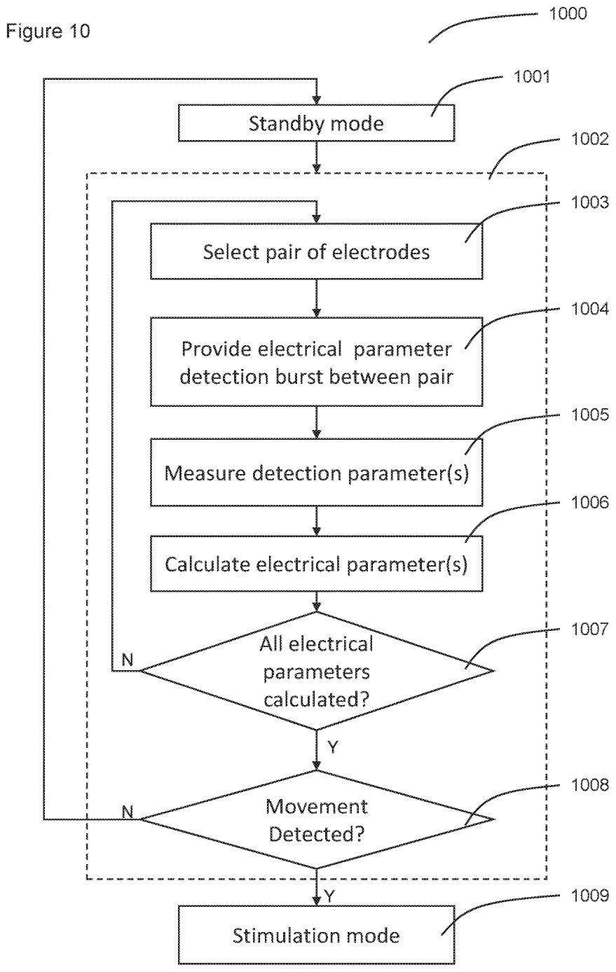

FIG. 10 shows a more detailed example embodiment of a method for providing electrical stimulation to a muscle of a user. The system is initially configured 1001 for an alternate mode, such as a standby mode. The controller 101 then provides signalling to the ES system 102 to operate the electrode array 104 in a detection mode 1002 during a detection period. The controller 101 may be triggered to enter a detection period by a timer, for example entering a detection period after 30 minutes in the standby mode.

While operating in the detection mode, the controller 101 selects 1003 a first pair of electrodes from the electrode array 104. The controller then provides signalling to cause the ES system 102 to provide 1004 a plurality of electrical parameter detection bursts during at least one detection period, wherein each electrical parameter detection burst comprises one or more detection pulses and wherein each electrical parameter detection burst is configured to not provide for stimulation of one or both of a muscle and a nerve at the target body location. The controller 101 detects and measures 1005 one or more detection parameters indicative of the response of the body associated with each electrical parameter detection burst. The controller then calculates 1006 one or more electrical parameters based on the one or more measured detection parameters.

Following (or concurrently with) the electrical parameter detection burst, the controller 101 selects a second pair of electrodes which may be any pair electrodes of the electrode array 104 except for the previously selected electrode pair, and steps 1004, 1005 and 1006 are repeated. Once the controller 101 evaluates 1007 that all electrical parameters required have been calculated, it can then analyse 1008 the electrical parameters to identify movement. High levels of change in the electrical parameters over time indicates movement, whereas low levels of change indicates inactivity.

If movement is detected, then the controller 101 may configure the system 100 to enter a standby mode and begin a timer to countdown to the next detection period. If movement is not detected, the controller may configure 1009 the system 100 into an alternate mode such as a stimulation mode. In the stimulation mode, the controller provides signalling causing the ES system to provide a plurality of stimulation pulses separated by rest periods wherein: each stimulation pulse comprises applying a stimulating potential difference between a first stimulation electrode and a second stimulation electrode of the electrode array such that the stimulating potential difference is configured to provide for stimulation at the target body location; and each rest period comprises a period of time which is absent of stimulation pulses.

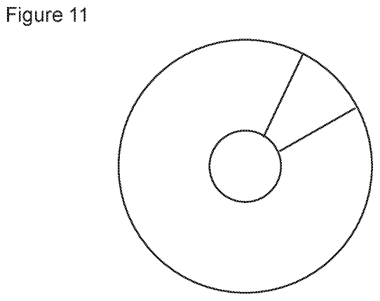

FIG. 11 shows a computer readable medium comprising computer program code configured to cause a controller comprising a processor and a memory to operate as described herein.

1. A controller for providing signalling to, and receiving signalling from, at least one electrical stimulation, ES, system, the ES system comprising an electrode array comprising a plurality of electrodes for applying an electrical stimulation to a muscle of a user and wherein the electrodes are further configured to measure an electrical parameter between any one electrode of the electrode array and any other one electrode of the electrode array, wherein the controller is configured to:
provide signalling to the ES system to operate the electrode array in a detection mode during a detection period, the signalling causing the ES system to provide a plurality of electrical parameter detection bursts, wherein each electrical parameter detection burst comprises one or more detection pulses and wherein each electrical parameter detection burst is configured to not provide for muscular stimulation; and
detect one or more detection parameters indicative of a degree of movement of the muscle associated with each electrical parameter detection burst,
wherein each electrical parameter detection burst is provided by applying a potential difference between different pairs of the plurality of electrodes, wherein a pair of electrodes refers to the combination of a first detection electrode and a second detection electrode selected from any electrode of the electrode array such that, when the controller provides a subsequent electrical parameter detection burst, the corresponding electrode pair for that subsequent electrical parameter detection burst comprises a pair of any two electrodes of the electrode array except for any electrode pair which would correspond to a previously provided electrical parameter detection burst of the detection period,
wherein the controller is configured to calculate the electrical parameter between each pair of detection electrodes based on the one or more detection parameters.

2. The controller of claim 1, wherein the controller is configured to provide signalling to the ES system to operate the electrode array in a stimulation mode, the signalling causing the ES system to provide a plurality of stimulation pulses separated by rest periods wherein:
each stimulation pulse comprises applying a stimulating potential difference between a first stimulation electrode and a second stimulation electrode of the electrode array such that the stimulating potential difference is configured to provide for stimulation of the muscle; and
each rest period comprises a period of time which is absent of stimulation pulses.

3. The controller of claim 2 wherein a detection period occurs during a rest period such that the one or more detection parameters are measured between the application of stimulation pulses.

4. The controller of any preceding claim wherein the controller is configured to provide signalling to the ES system to cause the ES system to provide an electrical parameter detection burst between at least 50% of the pairs of detection electrodes that can be defined within the electrode array and correspondingly detect one or more detection parameters indicative of the degree of movement of the body associated with each electrical parameter detection burst.

5. The controller of any preceding claim, wherein the controller is further configured to determine, based on the electrical parameters calculated between each pair of detection electrodes, whether to provide signalling to the ES system to operate the electrode array in the detection mode or in an alternate mode.

6. The controller of claim 1, wherein a detection parameter is the current between a pair of electrodes during the respective electrical parameter detection burst.

7. The controller of any preceding claim, wherein a calculated electrical parameter is at least one of:
an electrical resistivity;
an electrical resistance;
an electrical conductivity;
an electrical conductance;
an electrical impedance; and
an electrical admittance.

8. The controller of any preceding claim, wherein the controller is further configured to generate an electrical parameter map representative of a spatial distribution of the electrodes of the electrode array, wherein the electrical parameter map comprises a plurality of elements arranged in a grid and wherein each element comprises an element value based on one or more electrical parameters calculated by the controller.

9. The controller of claim 8, wherein each element of the electrical parameter map corresponds to an electrode of the electrode array, and the element value of an element of the electrical parameter map is based on a sum of each calculated electrical parameter that used the corresponding electrode.

10. The controller of claim 8, wherein each element of the electrical parameter map corresponds to an electrode of the electrode array, and the element value of an element of the electrical parameter map is based on an average of each calculated electrical parameter that used the corresponding electrode.

11. The controller of any of claims 8-10 wherein the controller is configured to obtain an electrical parameter map for electrodes of the electrode array obtained during a current detection period and wherein the element values of the electrical parameter map are based on a difference between element values calculated during the current detection period and one of:
an element value calculated during a preceding detection period taken at a time earlier than the current detection period; and
predefined element values.

12. The controller of claim 8, wherein each element of the electrical parameter map corresponds to a pair of electrodes of the electrode array, and the element value of an element of the electrical parameter is based on the difference between an electrical parameter calculated between the pair during the electrical parameter detection burst, and an electrical parameter calculated between the pair during a previous electrical parameter detection burst.

13. The controller of any of claims 8-11, wherein the controller is further configured to determine, based on the electrical parameter map, whether to provide signalling to the ES system to operate the electrode array in the detection mode or in an alternate mode.

14. The controller of any preceding claim, wherein the electrical parameter detection burst comprises a plurality of detection pulses each with a pulse frequency of between 10 Hz and 100 MHz.

15. The controller of claims 2-14, wherein the current supplied between each pair of detection electrodes for detection of a detection parameter is no more than 0.1 mA.

16. The controller of claim 2, wherein the controller is configured to, during the stimulation period, cause the ES system to apply a plurality of skin resistance reduction pulses, wherein the skin resistance reduction pulses comprise a higher frequency and a lower intensity compared to the plurality of stimulation pulses.

17. The controller of claim 16, wherein a plurality of the skin resistance reduction pulses are superimposed over a plurality of the stimulation pulses.

18. The controller of claims 16 or 17, wherein the skin resistance reduction pulses: carry a current of no more than 0.1 mA per pulse; and have a frequency between 1 and 100 000 Hz.

19. The controller of claim 2, wherein the controller is configured to, while operating in the stimulation mode, cause the ES system to apply a plurality of sensory blocking pulses, wherein the sensory blocking pulses have a lower intensity than the intensity of the stimulation pulses. 20, The controller of claims 19, wherein the sensory blocking pulses have a frequency between 0.1 and 150 Hz.

21. An electrical stimulation, ES, system for receiving signalling from and providing signalling to a controller of any of claims 1-20.

22. The ES system of claim 21 further comprising a garment wherein the garment provides a support for the relative arrangement of the electrodes in the electrode array and is configured to distribute at least the electrodes over at least a body part of the user.

23. A computer readable medium comprising computer program code configured to cause a controller to operate according to any of claims 1-22.

24. A method for providing electrical stimulation, ES, to a muscle of a user using at least one ES system, the ES system comprising a controller and an electrode array comprising a plurality of electrodes for applying the electrical stimulation to the muscle and wherein the electrodes are further configured to measure an electrical parameter between any one electrodes of the electrode array and any other one electrode of the electrode array, the method comprising the steps of:
the controller providing signalling to the ES system to operate the electrode array in a detection mode during a detection period, the signalling causing the ES system to provide a plurality of electrical parameter detection bursts during at least one detection period wherein each electrical parameter detection burst comprises one or more detection pulses and wherein each electrical parameter detection burst is configured to not provide for muscular stimulation; and
the controller detecting one or more detection parameters indicative of the degree of movement of the muscle associated with each electrical parameter detection burst, wherein each electrical parameter detection burst is provided by applying a potential difference between different pairs of the plurality of electrodes, wherein a pair of electrodes refers to the combination of a first detection electrode and a second detection electrode selected from any electrode of the electrode array such that, when the controller provides a subsequent electrical parameter detection burst, the corresponding electrode pair for that subsequent electrical parameter detection burst comprises a pair of any two electrodes of the electrode array except for any electrode pair which would correspond to a previously provided electrical parameter detection burst of the detection period, and the controller calculating the electrical parameter between each pair of detection electrodes based on the one or more detection parameters.

25. The method of claim 24, further comprising the step of:
the controller providing signalling to the ES system to operate the electrode array in a stimulation mode, the signalling causing the ES system to provide a plurality of stimulation pulses separated by rest periods wherein:
each stimulation pulse comprises applying a stimulating potential difference between a first stimulation electrode and a second stimulation electrode of the electrode array such that the stimulating potential difference is configured to provide for stimulation of the muscle; and
each rest period comprises a period of time which is absent of stimulation pulses.
